(19) United States
(12) Patent Application Publication
(10) Pub. No.: US 2026/0078041 A1
(43) Pub. Date: Mar. 19, 2026

(54) METHODS AND APPARATUSES FOR TRANSVERSE TEMPERATURE DISTRIBUTION DESIGN OF FORMING AND ANNEALING TO ENHANCE DRAW RATE

(71) Applicant: CAIHONG DISPLAY DEVICES CO., LTD., Xianyang (CN)

(72) Inventors: Menghu LI, Xianyang (CN); Hongrong HOU, Xianyang (CN); Yaoding CHAO, Xianyang (CN); Weidong HU, Xianyang (CN)

(73) Assignee: CAIHONG DISPLAY DEVICES CO., LTD., Xianyang (CN)

(21) Appl. No.: 19/397,825

(22) Filed: Nov. 21, 2025

Related U.S. Application Data
(63) Continuation-in-part of application No. PCT/CN2025/111231, filed on Jul. 29, 2025.

(30) Foreign Application Priority Data
Jul. 30, 2024 (CN) 202411033816.4

(51) Int. Cl. C03B 5/24; C03B 5/23; C03B 5/235; C03B 25/12

(52) U.S. Cl. CPC C03B 5/24; C03B 5/23; C03B 5/235; C03B 25/12

(57) ABSTRACT

Provided is a method and an apparatus for transverse temperature distribution design of forming and annealing to enhance a draw rate, which relates to the technical field of glass substrate manufacturing. The method includes dividing a forming and annealing process of a glass substrate into an overflow zone, a thickness forming zone, a pre-annealing zone, a soaking zone, an annealing zone, and a subsequent annealing zone based on six physical flow characteristics of glass in an overflow forming annealing zone. Meanwhile, differentiated transverse temperature distribution strategies are adopted in different temperature control zones to achieve precise control over the transverse temperature distribution design for glass forming and annealing. Additionally, the mechanism involved in the glass annealing process is deeply analyzed using “temperature difference-structural difference-thermal stress”, providing a more scientific and reasonable method for the warpage size and curved shape of glass after annealing and cooling to room temperature. It is particularly suitable for the temperature distribution design of the forming and annealing zone of glass substrates with a large draw rate, a wide plate width, and a thin profile.

### CROSS-REFERENCE TO RELATED APPLICATIONS

This application is a continuation-in-part application of International Application No. PCT/CN2025/111231, filed on Jul. 29, 2025, which claims priority to Chinese Patent Application No. 202411033816.4, filed on Jul. 30, 2024, the entire contents of each of which are hereby incorporated by reference.

### TECHNICAL FIELD

The present disclosure relates to the technical field of glass substrate manufacturing, and in particular to method and apparatus for transverse temperature distribution design of forming and annealing to enhance a draw rate.

### BACKGROUND

In the field of Thin Film Transistor Liquid Crystal Display (TFT-LCD) flat panel display manufacturing, the production of a glass substrate is crucial. The glass substrate manufactured by an overflow down-draw method has significant process and technical challenges among various manufacturing technologies. In particular, ensuring the thickness uniformity of the glass substrate, controlling residual stress, and controlling warpage are key elements of the process technology. These elements are closely related to the design and process control of the overflow forming annealing apparatus.

Warpage, as an important indicator for evaluating final product quality, arises from various causes. These causes include uneven heat distribution, improper viscosity control at the overflow brick tip, and interference from external forces. To effectively control warpage, the cooling curve from an overflow brick root to the strain point must be precisely regulated. Among these control measures, the control of the forming and annealing temperature is key to determining the cooling curve.

The forming annealing zone has the following characteristics. First, from top to bottom in the forming zone, the glass undergoes a hardening process. Second, during glass annealing, the edge region of the glass substrate generally dissipates heat faster than the central region. That is, a temperature difference exists between the edge region and the central region of the glass substrate. Third, the temperature difference between the edge region and the central region in the forming annealing zone needs to be reasonably controlled. The temperature difference between the edge region and the central region from the forming zone to the annealing zone should be kept as consistent as possible. This avoids edge distortion (edge warpage) caused by temperature difference imbalance. Therefore, in the design of the glass substrate annealing process, special attention needs to be paid to the hardening process of the glass from the forming zone to the annealing zone. Fine regulation of the temperature difference is also required. This ensures a balance of the temperature difference between the edge region and the central region of the glass substrate. Thus, edge distortion caused by the temperature difference can be avoided.

Within the traditional design framework of the glass substrate annealing process, designers have long relied primarily on the classical relaxation theory. This theory relatively simply covers three main physical states: a melt, a viscoplastic body, and an elastic body. Accordingly, the annealing process is roughly divided into a thickness forming stage, a slow annealing stage, and a rapid annealing stage. However, with continuous advancement in glass material science and thermal history research, an updated relaxation theory has emerged.

This new theory deeply analyzes the complex and variable physical behavior of glass during the cooling process. It finely divides the cooling process into six distinct physical characteristic stages. First is the free-flowing melt stage, where the glass is in a highly liquid state with strong fluidity. Next is the high viscoplastic body stage, where the viscosity increases significantly, but a certain plastic deformation capability is still maintained. Then comes the elasto-plastic body stage, where the glass begins to exhibit dual characteristics of elasticity and plasticity. After entering the initial state of the elastomer, its elastic response becomes more significant but has not yet reached a completely stable state. Subsequently, in the sub-rigid body stage, the physical characteristic of the glass gradually approaches rigidity, but certain internal relaxation activities are still retained. Finally, the glass is completely transformed into a rigid body. The physical characteristics of the glass tend to stabilize and no longer change significantly with temperature changes.

This division not only deepens the understanding of the physical state transition during the glass annealing process but also provides a more scientific basis for optimizing the annealing process and improving the quality of the glass substrate. By precisely controlling the cooling rate and temperature gradient in each stage, internal stress can be more effectively reduced, and structural uniformity can be optimized. Thus, glass products with superior performance can be produced. Furthermore, considering the need to strictly distinguish between irreversible structural differences and reversible structural differences, and between the narrow sense stress relaxation phenomenon and the broad sense stress relaxation phenomenon, viscosity (not just temperature) may be used as the dimension of the physical characteristic when discussing the division of the glass annealing zone.

Therefore, how to utilize viscosity and the six physical characteristic stages during the glass cooling process, to solve the problems of simple temperature distribution design in the existing forming annealing zone and imprecise warpage control, has become a technical problem urgently needing to be solved by those skilled in the art.

### SUMMARY

One or more embodiments of the present disclosure provide a method and apparatus for transverse temperature distribution design of forming and annealing to enhance a draw rate, to overcome the problems in the prior art of a simple design of temperature distribution in the forming annealing zone and imprecise warpage control caused by only considering three physical characteristics (i.e., the melt, the viscoplastic body, and the elastomer) based on the traditional relaxation theory.

The present disclosure solves the above technical problems through the following technical solutions:

A method for transverse temperature distribution design of forming and annealing to enhance a draw rate comprises: dividing an overflow forming annealing zone of an annealing furnace into a plurality of temperature control zones based on physical flow characteristics of glass in the overflow forming annealing zone in an updated relaxation theory, and in combination with glass viscosity corresponding to physical flow characteristics, types of the plurality of the temperature control zones comprising an overflow zone, a thickness forming zone, a pre-annealing zone, a soaking zone, an annealing zone, and a subsequent annealing zone; and applying different transverse temperature distribution design criterion to the overflow zone, the thickness forming zone, the pre-annealing zone, the soaking zone, the annealing zone, and the subsequent annealing zone, respectively.

The present disclosure also provides an apparatus for transverse temperature distribution design of forming and annealing to enhance a draw rate, configured to implement the method for transverse temperature distribution design of forming and annealing to enhance a draw rate described above.

### BRIEF DESCRIPTION OF THE DRAWINGS

The drawings are used to provide a further understanding of the present disclosure and constitute a part of the present disclosure. The illustrative embodiments of the present disclosure and the description thereof are used to explain the present disclosure and do not constitute an improper limitation of the present disclosure.

FIG. 1 is a schematic diagram illustrating an exemplary structure of an apparatus for transverse temperature distribution design of forming and annealing to enhance draw rate according to some embodiments of the present disclosure.

FIG. 2 is a schematic diagram illustrating an exemplary overflow down-draw glass according to some embodiments of the present disclosure.

FIG. 3 is a schematic diagram illustrating an exemplary temperature distribution design of a forming annealing zone according to some embodiments of the present disclosure.

In the figures, 1: overflow brick; 2: overflow trough; 3: molten glass feeding device; 4: overflow brick root; 5: flow guide plate; 6: formed glass substrate; 7: down-draw direction of the glass substrate; W_{G}: specification width of the glass substrate; W_{Y}: sheet guiding width of the glass substrate; and 8: overflow brick top.

### DETAILED DESCRIPTION

To make the objectives, technical solutions, and advantages of the embodiments of the present disclosure clearer, the technical solutions in the embodiments of the present disclosure are clearly and completely described below with reference to the accompanying drawings in the embodiments of the present disclosure. Obviously, the described embodiments are a part of the embodiments of the present disclosure, rather than all of the embodiments. Components of the embodiments of the present disclosure, which are generally described and illustrated in the accompanying drawings herein, can be arranged and designed in various configurations.

Therefore, the following detailed description of the embodiments of the present disclosure provided in the accompanying drawings is not intended to limit the scope of the claimed present disclosure, but merely represents selected embodiments of the present disclosure. All other embodiments obtained based on the embodiments in the present disclosure by those skilled in the art without making creative efforts fall within the protection scope of the present disclosure.

It should be noted that similar reference numerals and letters denote similar items in the following accompanying drawings. Therefore, once an item is defined in one accompanying drawing, it does not need to be further defined and explained in subsequent accompanying drawings.

In the description of the embodiments of the present disclosure, it should be noted that if terms such as “upper”, “lower”, “horizontal”, and “inner” are used to indicate orientations or positional relationships, the orientations or positional relationships are based on the orientations or positional relationships shown in the accompanying drawings, or the orientations or positional relationships in which the product of the present disclosure is conventionally placed when in use. These terms are used only to facilitate the description of the present disclosure and simplify the description, rather than to indicate or imply that the indicated apparatus or element must have a specific orientation or be constructed and operated in a specific orientation. Therefore, these terms should not be construed as limiting the present disclosure. In addition, terms such as “first” and “second” are used only for distinguishing description and should not be construed as indicating or implying relative importance.

In addition, if the term “horizontal” appears, it does not mean that the component must be absolutely horizontal, but may be slightly inclined. For example, “horizontal” merely means that its direction is more horizontal relative to “vertical” and does not mean that the structure must be completely horizontal, but may be slightly inclined.

In the description of the embodiments of the present disclosure, it should also be noted that, unless otherwise explicitly specified and defined, if terms such as “set”, “install”, “connected”, and “connect” appear, they should be understood in a broad sense. For example, the connection may be a fixed connection, a detachable connection, or an integral connection. The connection may be a mechanical connection, an electrical connection, or a direct connection. The connection may be a direct connection or an indirect connection through an intermediate medium. The connection may be an internal communication between two elements. Those skilled in the art may understand the specific meanings of the above terms in the present disclosure based on specific situations.

The following describes the present disclosure in further detail with reference to the accompanying drawings. The description is an explanation of the present disclosure rather than a limitation.

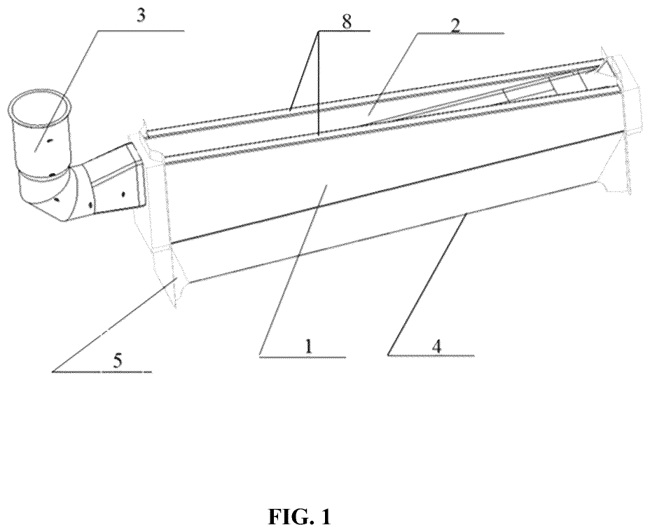

FIG. 1 is a schematic diagram illustrating an exemplary structure of an apparatus for transverse temperature distribution design of forming and annealing to enhance draw rate according to some embodiments of the present disclosure.

In some embodiments, as shown in FIG. 1, an apparatus for transverse temperature distribution design of forming and annealing to enhance draw rate includes an overflow brick 1, an overflow trough 2, a molten glass feeding device 3, an overflow brick root 4, and a flow guide plate 5.

The apparatus for transverse temperature distribution design of forming and annealing to enhance draw rate (also referred to as an overflow system) refers to an apparatus that uses a down-draw forming method to manufacture a glass substrate. As shown in FIG. 1, the overflow system includes an overflow brick 1 and a molten glass feeding device 3 that are connected to each other. An overflow brick refers to a refractory component used in the manufacturing process of the glass substrate. The molten glass feeding device is configured to provide molten glass material to be processed to the overflow brick.

In some embodiments, an overflow trough 2 is formed inside the overflow brick 1, and a bottom of the overflow brick 1 is the overflow brick root 4 (also referred to as an overflow brick tip). When the glass substrate is manufactured by a fusion overflow method, in a forming process, the molten glass melted by a glass melting furnace is supplied to the molten glass feeding device 3 in the apparatus for transverse temperature distribution design of forming and annealing to enhance draw rate, and flows along the overflow trough 2 to overflow from both sides of the overflow brick 1, thereby forming the glass substrate below the overflow brick root 4.

During the process in which the molten glass advances from a proximal end of the overflow trough to a distal end of the overflow trough, the molten glass overcomes laminar flow viscous resistance and advances under the action of a mass force and a pressure in a movement direction, and flows downward from an overflow weir. The overflow weir refers to an area formed at a top periphery of the overflow trough for downward overflow of the molten glass. The proximal end of the overflow trough refers to an end of the overflow trough close to the molten glass feeding device in a horizontal direction. The distal end of the overflow trough refers to an end of the overflow trough away from the molten glass feeding device in the horizontal direction. A fluid dynamics equation based on this principle integrates the effects of the above forces and is a basis for the design of the overflow trough.

On an overflow vertical surface, the mass force and the pressure acting on the molten glass are sufficiently large, while the viscosity of the molten glass is relatively low, so the influence of lateral surface tension is very small, resulting in almost no lateral shrinkage. On an overflow inclined surface, the components of the mass force and the pressure acting the molten glass along the inclined surface become significantly smaller, and the viscosity of the molten glass gradually increases, so the effect of the lateral surface tension becomes prominent, resulting in significant lateral shrinkage. Therefore, the flow guide plate 5 made of platinum material is provided at each of the proximal end and the distal end of the inclined surface of the overflow brick to partially resist the lateral shrinkage of the molten glass. The overflow vertical surface refers to an overflow surface on an upper section of the overflow brick that is vertical or substantially vertical. The overflow inclined surface refers to an inclined overflow surface on a lower section of the overflow brick.

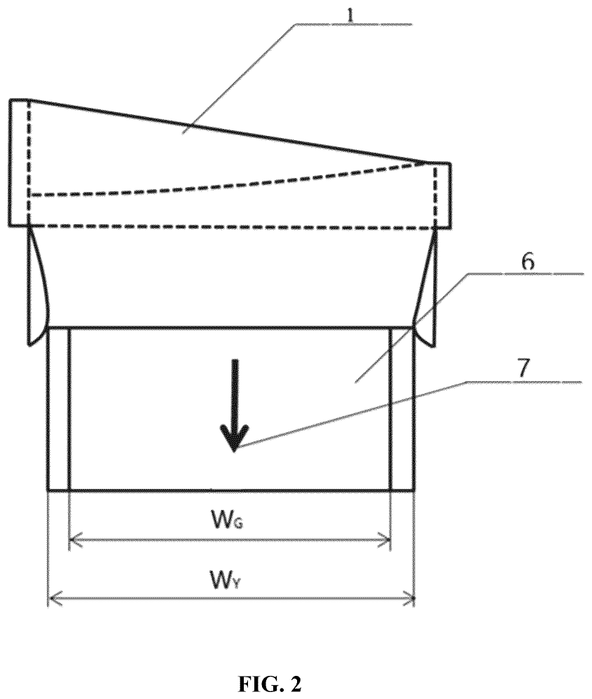

FIG. 2 is a schematic diagram illustrating an exemplary overflow down-draw glass according to some embodiments of the present disclosure.

As shown in FIG. 2, a sheet guiding is a basic action for forming the glass substrate. In the process of down-draw forming of the glass substrate, the formed glass substrate 6 moves downward along a down-draw direction 7 of the glass substrate. A moving direction in which the glass substrate moves downward is a flow direction (also referred to as a flowing direction). A non-flow direction (i.e., the direction from the proximal end to the distal end) is perpendicular to the flow direction. A transverse temperature direction described in some embodiments of the present disclosure refers to a direction along a specification width W_{G} of the glass substrate, which has the same meaning as the commonly used term “non-flow direction” in the industry.

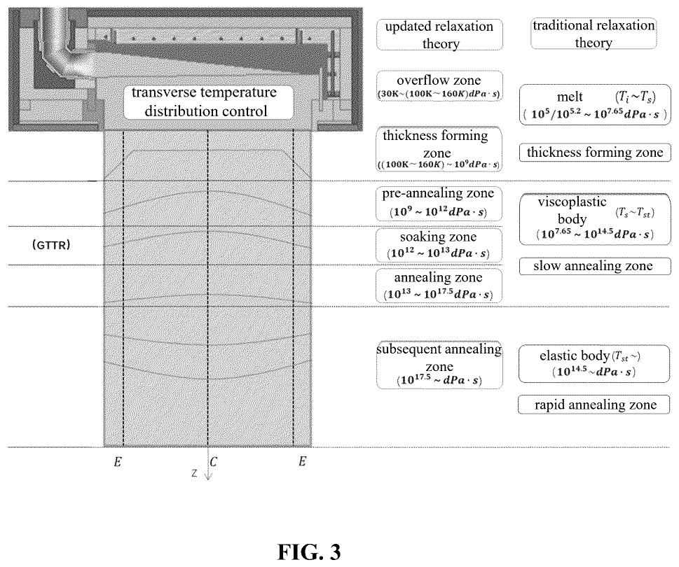

FIG. 3 is a schematic diagram illustrating an exemplary temperature distribution design of a forming annealing zone according to some embodiments of the present disclosure.

As shown in FIG. 3, a transverse temperature gradient between a central part and an edge part of the glass substrate is controlled to reduce residual stress, warpage, and a thickness extreme difference of the glass substrate. The central part refers to a central region of the glass substrate in the transverse direction, for example, a region C of the glass substrate as shown in FIG. 3. The edge part refers to regions on both sides of the glass substrate in the transverse direction, for example, regions E in FIG. 3. A maximum stress birefringence is 0.6 nm or less, a warpage value is 0.15 mm or less, and a thickness extreme difference is 10 μm to 15 μm or less. A non-flow direction temperature of the overflow brick tip may be uniformly controlled to be about 1150° C. (subject to actual process control specifications). The overflow brick tip is located at a tip portion of the overflow brick root and may cause the molten glass overflowing from both sides of the overflow brick to merge and form an initial glass substrate.

The traditional relaxation theory divides the annealing stage of the glass substrate into a thickness forming zone, a slow annealing zone, and a rapid annealing zone using three stages of “melt, viscoplastic body, and elastic body”, which cannot accurately reveal the mechanism of the annealing stage. Based on the six physical characteristic stages (i.e., free-flowing melt, high viscoplastic body, elasto-plastic body, initial state of elastomer, sub-rigid body, and rigid body), two annealing stages (i.e., a deformation-prevention annealing stage and a non-deformation annealing stage), and four annealing states (i.e., an optimal annealing state, a suboptimal annealing state, a least optimal annealing state, and a subsequent annealing state), the present disclosure obtains a technical route for the design of a glass annealing furnace, and uses the deduction of “temperature difference, structural difference, and thermal stress” to analyze the mechanism of glass annealing.

The deformation-prevention annealing stage refers to a stage in which the glass substrate is in a viscoelastic state and may deform. The non-deformation annealing stage refers to a stage in which the glass substrate is in an elastic solid state and does not deform. The pre-annealing zone belongs to the deformation-prevention annealing stage, and the glass substrate located in the pre-annealing zone is in the optimal annealing state. The soaking zone belongs to the deformation-prevention annealing stage, and the glass substrate located in the soaking zone is in the suboptimal annealing state. The annealing zone belongs to the non-deformation annealing stage, and the glass substrate located in the annealing zone is in the least optimal annealing state. The glass substrate in the subsequent annealing zone is in the subsequent annealing state.

A glass annealing theory may be established based on the hypothesis of the six physical characteristic stages to guide the design of the annealing furnace and annealing operations. Combined with the concept of plane stress, the causes of warpage, cracking, and cutting obstacles of the glass substrate (also referred to as a glass ribbon) may be analyzed. A discussion of the glass annealing furnace may use the viscosity (not only temperature) as a dimension of physical characteristics, strictly distinguish between irreversible structural differences and reversible structural differences, and distinguish between a narrow sense stress relaxation phenomenon and a broad sense stress relaxation phenomenon. According to updated research results on the thermal history of glass, the glass undergoes glassy states such as viscosity (e.g., free-flowing melt, high viscoplastic body), viscoelasticity (elasto-plastic body, initial state of elastomer, sub-rigid body), and elasticity (rigid body) during a cooling process of the glass. Different transverse temperature distributions are applied to the respective glassy state sections of the pre-annealing zone, the soaking zone, the annealing zone, and the subsequent annealing zone, to ensure that the glass has a stable curved surface shape after being annealed and cooled to room temperature.

The present disclosure provides a method for transverse temperature distribution design of forming and annealing to enhance a draw rate. The method may be executed by a processor. The method includes following operations.

Dividing an overflow forming annealing zone of an annealing furnace into a plurality of temperature control zones based on physical flow characteristics of glass in the overflow forming annealing zone in an updated relaxation theory and in combination with glass viscosity corresponding to the physical flow characteristics, wherein types of the plurality of temperature control zones comprise an overflow zone, a thickness forming zone, a pre-annealing zone, a soaking zone, an annealing zone, and a subsequent annealing zone.

Applying different transverse temperature distribution design criterion to the overflow zone, the thickness forming zone, the pre-annealing zone, the soaking zone, the annealing zone, and the subsequent annealing zone, respectively.

The forming annealing zone of the glass substrate is divided into the overflow zone, the thickness forming zone, the pre-annealing zone, the soaking zone, the annealing zone, and the subsequent annealing zone based on the updated relaxation theory of the glass substrate, starting from the six physical flow characteristics (i.e., the free-flowing melt, high viscoplastic body, elasto-plastic body, the initial state of elastomer, sub-rigid body, and the rigid body), and in combination with the glass viscosity.

The updated relaxation theory refers to a theoretical model that describes the physical state transition of glass during a cooling process. The updated relaxation theory considers that during the forming annealing process of the glass substrate, the physical flow characteristics of glass include the free-flowing melt, the high viscoplastic body, the elasto-plastic body, the initial state of elastomer, the sub-rigid body, and the rigid body, and in combination with the glass viscosity corresponding to the physical flow characteristics of glass, the overflow forming annealing zone is divided into the overflow zone, the thickness forming zone, the pre-annealing zone, the soaking zone, the annealing zone, and the subsequent annealing zone.

The overflow forming annealing zone refers to an entire spatial zone in a process of manufacturing the glass substrate using an overflow down-draw method, starting from overflow of molten glass on the overflow brick until the molten glass is cooled and solidified and leaves the annealing furnace. For example, the overflow forming annealing zone covers an entire glass substrate travel path from the overflow brick tip to an exit position of the annealing furnace.

The annealing furnace refers to a heat treatment device configured to perform controlled cooling on a formed glass substrate to eliminate or reduce internal stress. For example, in a production process adopting the overflow down-draw method, the annealing furnace refers to an elongated furnace body provided with different temperature zones along a travel direction of the glass substrate.

The physical flow characteristic refers to a macroscopic physical state and flow behavior characteristic exhibited by glass under different temperature and viscosity conditions.

The overflow zone refers to a zone in the overflow forming annealing zone where the glass mainly exists in a state of a free-flowing melt and flows from the overflow brick top to the overflow brick tip.

The thickness forming zone refers to a zone in the overflow forming annealing zone that follows the overflow zone, where the glass exhibits a state of the high viscoplastic body, and a thickness of the glass substrate is gradually formed and solidified through traction stretching.

The pre-annealing zone refers to a zone in the overflow forming annealing zone that is located after the thickness forming zone, where the glass exhibits a state of the elasto-plastic body, and preparation is made for subsequent formal annealing.

The soaking zone refers to a zone in the overflow forming annealing zone that is located after the pre-annealing zone, where the glass exhibits the initial state of elastomer, a temperature distribution inside the glass tends to be uniform, and conditions are created for effective stress relaxation.

The annealing zone refers to a zone in the overflow forming annealing zone that is located after the soaking zone, where the glass exhibits a state of the sub-rigid body, and permanent stress inside the glass is effectively eliminated through precise temperature control.

The subsequent annealing zone refers to a zone in the overflow forming annealing zone that is located after the annealing zone and extends to the exit position of the annealing furnace, where the glass exhibits a state of the rigid body, and a temporary stress is mainly generated.

The transverse temperature distribution design criterion refers to a temperature gradient controlling range set for controlling a temperature difference (ΔT) between a central region and an edge region of the glass substrate in different zones of the annealing furnace, such as the overflow zone, the thickness forming zone, and the pre-annealing zone. The criterion may reduce residual stress, suppress warpage, and ensure the thickness uniformity by precisely controlling the transverse temperature of the glass substrate.

In some embodiments, the transverse temperature distribution design of the glass substrate includes a transverse temperature distribution design of the overflow zone, a transverse temperature distribution design of the thickness forming zone, a transverse temperature distribution design of the pre-annealing zone, a transverse temperature distribution design of the soaking zone, a transverse temperature distribution design of the annealing zone, and a transverse temperature distribution design of the subsequent annealing zone.

In some embodiments, adopting differentiated transverse temperature distribution design criterion includes setting a temperature difference control target between the central region and the edge region for each temperature control zone.

According to some embodiments of the present disclosure, the forming annealing process of the glass substrate is divided into the overflow zone, the thickness forming zone, the pre-annealing zone, the soaking zone, the annealing zone, and the subsequent annealing zone based on six physical flow characteristics of glass in the overflow forming annealing zone, that is, the free-flowing melt, the high viscoplastic body, the elasto-plastic body, the initial state of elastomer, the sub-rigid body, and the rigid body. At the same time, differentiated transverse temperature distribution strategies are adopted in different annealing zones, thereby achieving precise control of the transverse temperature distribution design for glass forming annealing.

In some embodiments, when performing zone division of the overflow forming annealing zone, the following principles may be followed: the physical flow characteristic of glass in the overflow zone is the free-flowing melt, and a corresponding glass viscosity is in a range of 30K to 160K dPa·s; the physical flow characteristic of glass in the thickness forming zone is the high viscoplastic body, and a corresponding glass viscosity is in a range of 160K to 10⁹ dPa·s; the physical flow characteristic of glass in the pre-annealing zone is the elasto-plastic body, and a corresponding glass viscosity is in a range of 10⁹ to 10¹² dPa·s; the physical flow characteristic of glass in the soaking zone is the initial state of elastomer, and a corresponding glass viscosity is in a range of 10¹² to 10¹³ dPa·s; the physical flow characteristic of glass in the annealing zone is the sub-rigid body, and a corresponding glass viscosity is in a range of 10¹³ to 10^{17.5} dPa·s; and the physical flow characteristic of glass in the subsequent annealing zone is a rigid body, and a corresponding glass viscosity is at least 10^{17.5} dPa·s.

The free-flowing melt refers to a physical state in which the glass has a low viscosity and flows freely like a liquid. The high viscoplastic body refers to a physical state in which the glass has a high viscosity (e.g., 160K to 10⁹ dPa·s), flow is difficult, but plastic deformation still occurs under external force. The elasto-plastic body refers to a physical state in which the glass exhibits both elastic recovery and plastic deformation characteristics. The initial state of elastomer refers to a physical state in which elastic behavior of glass begins to dominate, but an internal structure of the glass is not yet completely stable. The sub-rigid body refers to a physical state in which physical properties of glass are close to those of a rigid body, but a certain internal stress relaxation capability is still retained. The rigid body refers to a physical state in which glass is completely transformed into a rigid solid, physical properties of the glass tend to be stable, and stress and strain are proportional and follow Hooke's law.

In some embodiments, when dividing zones based on the updated relaxation theory, the glass viscosity is used as a key parameter to define boundaries of the zones, and the viscosity value may be obtained through various measurement manners. The method of the embodiments of the present disclosure is applicable to all glass compositions that meet requirements of the overflow down-draw process, for example, low-alkali glass and alkali-free glass.

In some embodiments, since the subsequent annealing zone generates temporary stress, and the temporary stress is automatically eliminated due to temperature equalization of the glass substrate after the temperature of the glass substrate drops to room temperature, the temporary stress does not have a substantial effect on annealing stress. In some embodiments, to reduce a length of the entire apparatus for transverse temperature distribution design of forming and annealing to enhance draw rate, the subsequent annealing zone may be appropriately shortened, and a temperature at the exit position of the annealing furnace may be relatively higher, so an upper viscosity limit of the subsequent annealing zone may be determined based on a temperature design at the exit position of the annealing furnace.

In some embodiments, as shown in FIG. 3, a zone from the overflow brick top to the overflow brick tip may be determined as the overflow zone, a zone from the overflow brick tip to a theoretical annealing starting point may be determined as the thickness forming zone, a zone from the theoretical annealing starting point to an expansion softening point may be determined as the pre-annealing zone, a zone from the expansion softening point to an actual annealing starting point may be determined as the soaking zone, a zone from the actual annealing starting point to an actual annealing lower limit point may be determined as the annealing zone, and a zone from the actual annealing lower limit point to the exit position of the annealing furnace may be determined as the subsequent annealing zone.

The theoretical annealing starting point refers to a temperature point at which the glass substrate theoretically transforms from a high viscoplastic body to an elasto-plastic body and begins to exhibit measurable structural relaxation behavior during the annealing process. The expansion softening point refers to a temperature point at which the glass undergoes temporary volume expansion due to structural relaxation when the glass substrate transitions from the elasto-plastic body to the initial state of elastomer during the annealing process. The actual annealing starting point refers to a starting temperature point at which the glass substrate transitions from the initial state of elastomer to the sub-rigid body and begins to enter an actual annealing stage primarily aiming at eliminating permanent stress during the annealing process. The actual annealing lower limit point refers to a temperature point at which permanent stress of the glass substrate has been substantially eliminated during the annealing process, marking an end of an effective annealing stage.

In some embodiments, a glass viscosity in a range of 100K to 160K dPa·s is a viscosity ηᵢ of the glass substrate at the overflow brick tip; 10⁹ dPa·s is a viscosity η_{T} of the glass substrate at the theoretical annealing starting point; 10¹² dPa·s is a viscosity η_{d} of the glass substrate at the expansion softening point; 10¹³ dPa·s is a viscosity ηₐ of the glass substrate at the actual annealing starting point; 10^{17.5} dPa·s is a viscosity nae of the glass substrate at the actual annealing lower limit point, and the viscosity ηₐₑ of the glass substrate at the actual annealing lower limit point is greater than a viscosity ηₛₜ=10^{14.5} dPa·s of the glass substrate at the theoretical annealing lower limit point. The theoretical annealing lower limit point refers to a strain point at which the glass substrate theoretically transitions completely from an elastomer to a rigid body during the annealing process.

According to the above zone division, in combination with a principle of glass overflow forming: molten glass flows through an overflow trough of the overflow brick, bypasses an overflow weir and flows downward along overflow surfaces on both sides of the overflow brick, merges at the overflow brick tip, and is cooled and formed by means of traction stretching. Different transverse temperature distribution design criterion is applied to the overflow zone, the thickness forming zone, the pre-annealing zone, the soaking zone, the annealing zone, and the subsequent annealing zone, thereby controlling thickness uniformity of the obtained glass substrate after annealing and satisfying certain warpage requirements.

The traction stretching refers to a continuous downward pulling force applied to a glass substrate flowing down from the overflow brick tip by an external device (e.g., a traction roller device).

According to some embodiments of the present disclosure, the down-draw forming process of the glass substrate is divided in more detail into the overflow zone, the thickness forming zone, the pre-annealing zone, the soaking zone, the annealing zone, and the subsequent annealing zone based on physical flow characteristics of the molten glass in different periods and corresponding viscosity ranges, which allows for more precise temperature control of the glass substrate during the down-draw forming process and is more conducive to reducing the residual stress, the warpage, and the thickness extreme difference of the glass substrate during the forming process.

The transverse temperature distribution design of overflow zone includes that a temperature range from the overflow brick top to the overflow brick tip is 1240° C. to 1155° C.

The overflow brick top refers to a top region of the overflow brick, i.e., a starting position where the molten glass overflows from the overflow trough and flows downward along both sides of the overflow brick. In the overflow zone, the physical flow characteristic of glass is the free-flowing melt, and a corresponding glass viscosity is in a range of 30K to 160K dPa·s. Considering that the viscosity of glass in the overflow zone is low and it is a flowing body, the transverse temperature distribution design is performed only for the overflow brick tip in the overflow zone.

In some embodiments, since heat dissipation of platinum material flow guide plate at proximal end and distal end is faster, an edge region E has a lower temperature than a central region C. A reduction amount of a temperature T_{1E} of the edge region E relative to a temperature T_{1C} of the central region C is controlled to be less than or equal to 20° C. That is, the transverse temperature distribution design criterion of the overflow zone includes: based on the physical flow characteristic of the glass in the overflow zone being the free-flowing melt, the transverse temperature distribution of the overflow brick tip of the overflow zone being designed to satisfy a formula (1):

$$0 ° C . \leq Δ T 1 = T {1}_{C} - T {1}_{E} \leq 20 ° C . , \qquad ( 1 )$$

In the formula (1), T_{1E} is an edge temperature of the overflow brick tip of the overflow zone; T_{1C} is a central temperature of the overflow brick tip of the overflow zone; and ΔT1 is a transverse temperature difference of the overflow brick tip of the overflow zone. The central temperature and the edge temperature of the overflow brick tip may be obtained by a temperature detector, for example, an infrared thermometer.

At the same time, it is necessary to ensure that the edge temperature T_{1E} is not lower than an upper crystallization temperature T_{XU}. The upper crystallization temperature T_{XU} of the glass is determined based on a liquidus temperature. The upper crystallization temperature of the glass refers to a highest temperature limit at which a crystalline phase begins to precipitate during the cooling process of the glass, below which a risk of crystallization significantly increases. The upper crystallization temperature T_{XU}=T_{L}−(12 to 22° C.); T_{L} is the liquidus temperature, which is a highest temperature at which the crystalline phase and the glass coexist in equilibrium, and the liquidus temperature T_{L} is less than a temperature T_{160K} when a glass viscosity is 160K dPa·s. The glass with a higher viscosity (e.g., a viscosity greater than 160K dPa·s) will grow unacceptable devitrification (crystal) defects, affecting a forming stability of the glass substrate, and an overflow fusion requires a high liquidus viscosity to minimize a crystallization devitrification of a finished glass.

In some embodiments, the transverse temperature distribution design of the overflow zone may be achieved by adjusting a heating element or a thermal insulation structure near the overflow brick tip. For example, the heating element may be arranged at a central region and an edge region of the overflow brick tip to independently control the temperature. A person skilled in the art may understand that the temperature control of the overflow zone is not limited to the above method, and other methods capable of achieving a uniform distribution of the transverse temperature fall within a protection scope of the present disclosure.

According to some embodiments of the present disclosure, the transverse temperature distribution of the overflow zone at the overflow brick tip is designed to range from 0° C. to 20° C., and an edge temperature of the overflow brick tip is also limited to be not lower than the upper crystallization temperature. On a basis of ensuring a precise forming of the glass substrate, the devitrification defect of the glass substrate is prevented, and a forming quality of the glass substrate is more accurately ensured.

The physical flow characteristic of the glass in the thickness forming zone is the high viscoplastic body, and a corresponding glass viscosity is in a range of 160K to 10⁹ dPa·s. The transverse temperature distribution design of the thickness forming zone includes as follows.

Since cooling by edge rollers causes a non-flow direction temperature (i.e., the transverse temperature) of the glass substrate to rapidly decrease from the central region C to edge regions E, and the temperature reduction at the edge regions E where the edge rollers are located is the most significant compared to the central region C. Therefore, it is necessary to inhibit a non-flow direction shrinkage of the glass substrate to ensure a sufficient sheet guiding width and a sheet guiding stability.

However, the central region C (excluding edge regions) basically maintains a uniform temperature that is relatively higher than the edge regions E. A range of a transverse temperature difference ΔT2_{C} of the central region of the thickness forming zone is set to 0° C. to 20° C., so as to control a uniform temperature distribution of the central region of the thickness forming zone to ensure uniform thickness distribution of the glass substrate and reduce the thickness extreme difference.

In some embodiments, from the position of the edge rollers to the theoretical annealing starting point, a temperature difference between the central temperature T2_{C} and an edge temperature T2_{E} gradually decreases, and a glass thickness is basically finalized to a target thickness. As the drawn rate increases and a sheet guiding speed accelerates, the thickness forming zone requires more cooling to accelerate the glass thickness formation, and at the same time, provides more space for a subsequent glass transition temperature region (GTTR) to cope with an increased flow rate and reduce a reheating shrinkage.

That is, the transverse temperature distribution design of the thickness forming zone includes: designing the transverse temperature distribution of the thickness forming zone along the flow direction of the glass substrate to satisfy a formula (2):

$$120 ° C . \leq Δ T 2 = T {2}_{C} - T {2}_{E} \leq 190 ° C . , \qquad ( 2 )$$

In the formula (2), T2_{E} is an edge temperature of the thickness forming zone; T2_{C} is a central temperature of the thickness forming zone; ΔT2 is a transverse temperature difference of the thickness forming zone along the flow direction.

In some embodiments, the transverse temperature distribution design of the thickness forming zone is achieved by setting an additional cooling device at the position of the edge rollers. For example, the cooling device may include an air cooling system or a water cooling system to accelerate the edge region cooling. Those skilled in the art may understand that a temperature control of the thickness forming zone is not limited to the cooling device, and a temperature distribution may be optimized by adjusting a position or a speed of the edge rollers.

According to some embodiments of the present disclosure, based on the physical flow characteristic of the glass in the thickness forming zone and the corresponding viscosity range, and considering a rapid decrease in the edge temperature, the transverse temperature distribution of the thickness forming zone is designed to a range of 120° C. and 190° C., and the transverse temperature difference ΔT2_{C} of the central region is maintained at a uniform distribution of 0° C. to 20° C. A larger overall temperature difference effectively inhibits an excessive shrinkage of the glass substrate in a width direction, ensuring a sufficient sheet guiding width and a stability of a forming process. Meanwhile, a precise temperature uniformity control of the central region directly contributes to a high-precision uniform thickness distribution of the glass substrate, significantly reducing the thickness extreme difference.

The physical flow characteristic of the glass in the pre-annealing zone is the elasto-plastic body, and a corresponding glass viscosity is in a range of 10⁹ to 10¹² dPa·s. The transverse temperature distribution design of the pre-annealing zone includes as follows.

The pre-annealing zone ranges from the theoretical annealing starting point of the glass substrate to an expansion softening point. Referring to FIG. 3, a non-flow direction temperature (a transverse temperature) distribution of the pre-annealing zone gradually transitions from a uniform distribution at the central region of the thickness forming zone to a nearly parabolic distribution that gradually decreases from the central region C to the edge regions E of the glass substrate. The pre-annealing zone includes a cooling unit, and thermodynamic and kinetic characteristics are structural group displacement and molecular displacement, with a manifested structural relaxation of 0. The cooling unit is configured to cool the formed glass substrate, for example, the air cooling device and the water cooling device. Although an annealing behavior theoretically exists, a main purpose is still to maintain a large temperature difference to provide a prerequisite for subsequent soaking and annealing.

In some embodiments, the transverse temperature distribution design criterion of the pre-annealing zone includes as follows.

A temperature difference between a central temperature and an edge temperature of the pre-annealing zone gradually decreasing from a theoretical annealing starting point of the glass substrate to an expansion softening point along a flow direction of the glass substrate, and the transverse temperature distribution at the expansion softening point is designed to satisfy a formula (3):

$$35 ° C . \leq Δ T 3 = T {3}_{C} - T {3}_{E} \leq 50 ° C . , \qquad ( 3 )$$

In the formula (3), a junction position between the thickness forming zone and the pre-annealing zone is the theoretical annealing starting point of the glass substrate, and a junction position between the pre-annealing zone and a soaking zone is the expansion softening point of the glass substrate. T3_{E} is the edge temperature of the pre-annealing zone. T3_{C} is the central temperature of the pre-annealing zone. ΔT3 is a transverse temperature difference at the expansion softening point. The central temperature and the edge temperature of the pre-annealing zone may be obtained by the temperature detector, for example, the infrared thermometer.

In some embodiments, the transverse temperature distribution design of the pre-annealing zone is achieved by setting a zoned heating system in the pre-annealing zone. For example, the heating system may independently control the temperature of the central region and the edge regions of the glass substrate to gradually reduce the temperature difference. Those skilled in the art may understand that a temperature control of the pre-annealing zone is not limited to the heating system, and a temperature gradient may be adjusted by combining the cooling unit.

According to some embodiments of the present disclosure, by precisely controlling the transverse temperature difference ΔT3 of the pre-annealing zone at the expansion softening point within a range of 35° C. to 50° C., this temperature range effectively induces an orderly relaxation and adjustment of an internal structure of the glass in an elasto-plastic state, providing full preparation for subsequent soaking and annealing processes. In addition, the design of this temperature range pre-releases a portion of thermal stress, preventing deformation caused by a drastic temperature change.

The physical flow characteristic of glass in the soaking zone is the initial state of elastomer, and a corresponding glass viscosity is in a range of 10¹² to 10¹³ dPa·s. The transverse temperature distribution design of the soaking zone includes as follows.

From the expansion softening point to an actual annealing starting point of the glass substrate, a non-flow direction temperature gradient formed in the pre-annealing zone gradually decreases toward the actual annealing starting point, and an overall very low vertical cooling rate is maintained from the expansion softening point to the actual annealing starting point. The soaking zone corresponds to a glass transition region in a fusion overflow down-draw method, and the thermodynamic and the kinetic characteristics are that a structural group displacement tends toward molecular displacement. A sharp increase in viscosity causes a displacement activity and a differential deformation activity of the glass substrate to decrease sharply. Since a thermal conductivity coefficient slightly decreases with a temperature drop, even if a constant lower cooling rate is maintained, a thermal stress generated by a structural relaxation has become manifestly measurable. A non-flow direction temperature of the glass substrate (also referred to as a flat glass) is a temperature region from below the expansion softening point of the glass to the actual annealing starting point. An absolute value of a temperature difference between the edge regions E and the central region C tends to be small along the flow direction. The soaking zone is a most effective region for controlling a thermal shrinkage and the warpage of the glass substrate.

In some embodiments, the transverse temperature distribution design criterion of the soaking zone includes as follows.

A temperature difference between a central temperature and an edge temperature of the soaking zone gradually decreases from an expansion softening point of a glass substrate to an actual annealing starting point along a flow direction of the glass substrate, and the transverse temperature distribution at the actual annealing starting point is designed to satisfy a formula (4):

$$17.5 ° C . \leq Δ T 4 = T {4}_{C} - T {4}_{E} \leq 25 ° C . , \qquad ( 4 )$$

In the formula (4), a junction position between the pre-annealing zone and the soaking zone is the expansion softening point of the glass substrate, a junction position between the soaking zone and the annealing zone is the actual annealing starting point of the glass substrate, T4_{E} is the edge temperature of the soaking zone; T4_{C} is the central temperature of the soaking zone; and ΔT4 is a transverse temperature difference at the actual annealing starting point. The central temperature and the edge temperature of the soaking zone may be obtained by the temperature detector, for example, the infrared thermometer.

In some embodiments, the transverse temperature distribution design of the soaking zone may be achieved by adopting a uniform heating manner in the soaking zone to reduce the temperature difference. For example, the uniform heating may be implemented by arranging a plurality of heating elements and controlling a power output thereof. Those skilled in the art may understand that temperature control of the soaking zone is not limited to heating methods and may also optimize thermal uniformity by adjusting a travel speed of the glass substrate.

According to some embodiments of the present disclosure, by designing the soaking zone such that a temperature difference between the central region and the edge region gradually converges from the expansion softening point to the actual annealing starting point, and precisely controlling the transverse temperature difference ΔT4 at the actual annealing starting point within a small range of 17.5° C. to 25° C., a transverse temperature gradient can be smoothly and uniformly achieved at a low vertical cooling rate, allowing the glass to fully and uniformly release its internal structural stress at the key stage of the initial state of elastomer. This fine control not only minimizes warpage deformation caused by uneven thermal shrinkage and stress concentration, but also lays a foundation for a stress-free state in the subsequent annealing zone, ensuring effective control of the glass substrate to obtain high flatness, low residual stress, and a stable curved shape.

The physical flow characteristic of the glass in the annealing zone is the sub-rigid body, and a corresponding glass viscosity is in a range of 10¹³ to 10^{17.5} dPa·s. The transverse temperature distribution design of the annealing zone includes as follows.

From the actual annealing starting point to the actual annealing lower limit point of the glass substrate, a non-flow direction temperature (transverse temperature) gradient formed in the pre-annealing zone further decreases toward the actual annealing lower limit point. A low vertical cooling rate is maintained from the actual annealing starting point to the actual annealing lower limit point, further reducing the annealing stress, the warpage, and the thermal shrinkage. The annealing zone has transitioned to a completely elastic body (i.e., the sub-rigid body) stage. Although it exhibits non-deformable annealing, it is the least effective annealing state compared to the soaking zone and the pre-annealing zone, corresponding to a stress control zone in a fusion overflow down-draw method. Thermodynamic and kinetic characteristics involve molecular displacement. Although it has not yet reached a degree where stress is proportional to strain and follows Hooke's law, it already exhibits characteristics of the sub-rigid body. The temperature gradient of the non-flow direction temperature of the glass substrate from the edge region to the central region tends to be minimized. A cooling rate of a central region of the glass substrate is relatively fast, forming tensile stress in both a flow direction and a non-flow direction of the central region of the glass substrate, and the tensile stress in the flow direction is much greater than the tensile stress in the non-flow direction, thereby reducing the warpage of the glass substrate.

In some embodiments, the transverse temperature distribution design criterion of the annealing zone includes as follows.

A temperature difference between a central temperature and an edge temperature of the annealing zone gradually decreases from an actual annealing starting point of a glass substrate to an actual annealing lower limit point along the flow direction of the glass substrate. The transverse temperature distribution at the actual annealing lower limit point is designed to satisfy a formula (5):

$$0 ° C . \leq Δ T 5 = T {5}_{C} - T {5}_{E} \leq 15 ° C . , \qquad ( 5 )$$

In the formula (5), a junction position between the soaking zone and the annealing zone is the actual annealing starting point of the glass substrate. A junction position between the annealing zone and the subsequent annealing zone is the actual annealing lower limit point of the glass substrate. T5_{E} is the edge temperature of the annealing zone. T5_{C} is the central temperature of the annealing zone. ΔT5 is a transverse temperature difference at the actual annealing lower limit point. The central temperature and the edge temperature of the annealing zone may be obtained by the temperature detector, e.g., the infrared thermometer.

In some embodiments, the transverse temperature distribution design of the annealing zone may be achieved by arranging a low-speed cooling system in the annealing zone. For example, the low-speed cooling system may control cooling rate of the central region and the edge region to minimize the temperature difference. Those skilled in the art may understand that temperature control of the annealing zone is not limited to the cooling system, and the temperature uniformity may also be maintained through thermal shielding or other insulation measures.

According to some embodiments of the present disclosure, by designing the annealing zone to maintain a low vertical cooling rate from the actual annealing starting point to the actual annealing lower limit point, and controlling the transverse temperature difference ΔT5 at the actual annealing lower limit point within a low temperature difference range of 0° C. to 15° C., in the stage where the glass has transformed into a sub-rigid body and only undergoes non-deformable annealing, generation of new thermal stress is greatly suppressed by minimizing the transverse temperature gradient, and residual permanent stress inside the glass is allowed to fully and uniformly relax. The warpage caused by uneven stress is effectively avoided.

The physical flow characteristic of the glass in the subsequent annealing zone is the rigid body, and a corresponding glass viscosity is at least 10^{17.5} dPa·s. The transverse temperature distribution design of the subsequent annealing zone includes as follows.

From the actual annealing lower limit point of the glass substrate to a cutting position where the glass substrate is naturally cooled to room temperature, a non-flow direction temperature gradient formed in the subsequent annealing zone (also referred to as a transverse temperature difference at an exit position of the annealing furnace) ΔT6=T6_{C}−T6_{E} further decreases toward the exit position of the annealing furnace, even forming a small reverse temperature gradient, and finally naturally cooling to a uniform room temperature. The cutting position refers to a position where the glass substrate is cut into a product of a specific size. For example, the cutting position may be located at or outside the exit position of the annealing furnace. The subsequent annealing zone maintains a high vertical cooling rate, which has no practical annealing significance for inherent annealing stress, warpage, and thermal shrinkage. The subsequent annealing zone is already in a completely elastic body (i.e., the rigid body) stage. The thermodynamic and kinetic characteristics involve particle vibration, and stress is proportional to strain and follows Hooke's law. Structural differences of the glass substrate are fully revealed until the temperature becomes uniform room temperature and no longer changes, and the thermal stress becomes permanent stress. A temperature difference of the glass substrate in the subsequent annealing zone only produces a reversible structural difference because there is no differential deformation. Such structural difference is fully revealed, and the thermal stress that disappears as the temperature becomes uniform is called a temporary stress. The temporary stress superimposes with the permanent stress at vector coincidence points before the temporary stress disappears. Cracking occurs when a single stress or a superimposed stress exceeds a tensile strength of the glass.

In some embodiments, the transverse temperature distribution design criterion of the subsequent annealing zone includes as follows.

A temperature difference between a central temperature and an edge temperature of the subsequent annealing zone gradually decreases from an actual annealing lower limit point of a glass substrate to an exit position of the annealing furnace along a flow direction of the glass substrate. The transverse temperature distribution at the exit position of the annealing furnace is designed to satisfy a formula (6):

$$- 35 ° C . \leq Δ T 6 = T {6}_{C} - T {6}_{E} \leq 0 ° C . , \qquad ( 6 )$$

In the formula (6), a junction position between the annealing zone and the subsequent annealing zone is the actual annealing lower limit point of the glass substrate. T6_{E} is the edge temperature of the subsequent annealing zone. Toc is the central temperature of the subsequent annealing zone. ΔT6 is a transverse temperature difference at the exit position of the annealing furnace. The central temperature and the edge temperature of the subsequent annealing zone may be obtained by the temperature detector, e.g., the infrared thermometer.

In some embodiments, the transverse temperature distribution design of the subsequent annealing zone may be achieved by arranging a reverse heating device at the exit position. For example, the reverse heating device may preferentially heat the edge region of the subsequent annealing zone to generate a negative temperature difference. Those skilled in the art may understand that temperature control of the subsequent annealing zone is not limited to the heating device, and a desired temperature distribution may also be achieved by adjusting environmental cooling conditions.

According to some embodiments of the present disclosure, by designing the subsequent annealing zone to form a small reverse temperature gradient (i.e., the edge temperature being slightly higher than the central temperature) of −35° C. to 0° C. at the exit position of the annealing furnace, in the stage where the glass has completely transformed into the rigid body, a distribution of temporary thermal stress of the glass substrate during a final cooling stage is balanced through mild reverse temperature compensation. This effectively prevents a risk of cracking caused by local superposition of temporary stress and permanent stress, while ensuring that the glass substrate can smoothly transition to room temperature after leaving the annealing furnace, ultimately obtaining stable and uniform physical characteristics.

In some embodiments, the method for transverse temperature distribution design of forming and annealing to enhance a draw rate is a design method of real-time transverse temperature distribution during a glass forming process. The transverse temperature distribution design criterion includes a target temperature difference range. The method further includes: for each of the plurality of temperature control zones, determining a type of the overflow forming annealing zone to which the temperature control zone belongs; determining target temperature difference ranges corresponding to the plurality of temperature control zones according to the transverse temperature distribution design criterion corresponding to types of overflow forming annealing zones to which the plurality of temperature control zones belong; and controlling a cooling array to spray a cooling gas, and/or controlling a heating array to heat according to the target temperature difference ranges corresponding to the plurality of temperature control zones, to control a temperature difference between a central temperature and an edge temperature in each of the plurality of temperature control zone within the target temperature difference range corresponding to each of the plurality of temperature control zone.

The target temperature difference range refers to a temperature difference range between a central temperature and an edge temperature required by different temperature control zones (e.g., the overflow zone and the thickness forming zone). For example, the target temperature difference range of the overflow zone is in a range of 0° C. to 20° C. Determination of the target temperature difference range for each temperature control zone may refer to the relevant descriptions of the transverse temperature distribution design criterion for the plurality of temperature control zones (i.e., the overflow zone, the thickness forming zone, the pre-annealing zone, the soaking zone, the annealing zone, and the subsequent annealing zone).

The cooling array refers to a cooling device for cooling the glass substrate, including a plurality of cooling units arranged along an axial direction of the annealing furnace, e.g., gas nozzles.

The cooling gas refers to a gas used for spraying to cool the glass substrate, e.g., nitrogen and helium.

The heating array refers to a heating device for heating the glass substrate, including a plurality of heating units arranged along the axial direction of the annealing furnace, e.g., infrared lamp tubes.

In some embodiments, the processor may control the heating array and/or the cooling array to heat and/or cool each temperature control zone according to the target temperature difference range corresponding to each temperature control zone.

In some embodiments, the controlling the cooling array to spray the cooling gas, and/or controlling the heating array to heating includes: for each of the plurality of temperature control zones, generating an optimal temperature control instruction through a static instruction library according to a molten glass temperature, a material characteristic, a down-draw parameter, and the target temperature difference range every preset period, wherein the optimal temperature control instruction controls the plurality of heating units in the heating array corresponding to the plurality of temperature control zones to heat at an optimal heating power during an optimal heating period, and/or controls a plurality of cooling units in the cooling array in the plurality of temperature control zones to spray the cooling gas at an optimal valve opening during an optimal cooling period.

The preset period refers to a period for adjusting a temperature of the glass substrate for each temperature control zone, e.g., 10 s or 30 s.

In some embodiments, the preset period may be set by a technician based on experience.

In some embodiments, preset periods corresponding to the plurality of temperature control zones may be the same or different.

The molten glass temperature refers to a current temperature of the molten glass in each temperature control zone.

In some embodiments, the molten glass temperature may be obtained by the temperature detector, e.g., the infrared thermometer.

The material characteristic refers to a physical property of the molten glass in a corresponding temperature control zone, including thermal conductivity, specific heat capacity, viscosity, etc.

In some embodiments, the material characteristic of the molten glass may be obtained through factory parameters of glass raw materials or based on testing.

The down-draw parameter refers to a process parameter during a down-draw forming process of the glass, including a down-draw speed, a target thickness, and a glass width.

The down-draw speed refers to a sheet guide speed of the glass during the down-draw forming process. The target thickness refers to a required final thickness of the glass substrate. The glass width refers to the specification width of the glass substrate.

More descriptions regarding the sheet guide speed and the specification width of the glass substrate can be found in FIG. 2 and FIG. 3 and related descriptions thereof.

The static instruction library refers to a database corresponding to each temperature control zone and containing a plurality of temperature control instructions. The static instruction library stores optimal control instructions required for achieving the target temperature difference range under different working conditions for each temperature control zone.

The working condition refers to a process situation during the down-draw forming process of the glass, including the molten glass temperature, the material characteristic, the down-draw parameter, etc.

In some embodiments, each temperature control zone may correspond to one static instruction library.

In some embodiments, the static instruction library may be obtained through cluster analysis.

In some embodiments, the processor may construct clustering vectors based on the molten glass temperature, a historical material characteristic, and an adopted historical down-draw parameter during multiple historical annealing processes for each temperature control zone, and a historical target temperature difference range corresponding to the temperature control zone, and use a historical temperature control instruction actually adopted during the multiple historical annealing processes (hereinafter referred to as the historical temperature control instruction) as a label of the clustering vectors. The processor performs clustering on the clustering vectors to obtain a plurality of clusters. For each cluster in the plurality of clusters, a corresponding working condition range determined based on the molten glass temperature, the material characteristic, and the down-draw parameter of all clustering vectors in the cluster and a corresponding target temperature difference range are used as a piece of reference data in the static instruction library. A mean value of top N historical temperature control instructions with a best temperature control effect among the historical temperature control instructions corresponding to all clustering vectors in the cluster corresponding to the reference data is used as a reference optimal temperature control instruction. N is a value greater than 0 and may be set by a technician based on experience.

In some embodiments, the best temperature control effect includes: for each temperature control zone, a time required to achieve the target temperature difference range is shortest, and during the process of achieving the target temperature difference range, an amplitude and a frequency of temperature fluctuations of the temperature control zone are smallest.

In some embodiments, the temperature control effect of the historical temperature control instruction may be obtained based on historical data or experimental data.

The optimal temperature control instruction refers to a parameter combination for controlling heating array and/or cooling array that is matched according to a current real-time working condition of the temperature control zone and most suitable for each temperature control zone, including a count of heating units and/or a count of cooling units, the optimal heating period, the optimal heating power, the optimal cooling period, the optimal valve opening, etc. For example, the optimal temperature control instruction includes: two cooling units located in the overflow zone with serial numbers “1 #” and “2 #”, a valve opening of 30°, a spray duration of 10 s, etc.

In some embodiments, the processor may obtain a current real-time working condition and the target temperature difference range of each temperature control zone and determine the optimal temperature control instruction. For example, the processor may perform matching in the static instruction library based on the current real-time working condition and the target temperature difference range of each temperature control zone, and take a reference optimal temperature control instruction corresponding to a reference data that best matches the current real-time working condition and the target temperature difference range of the temperature control zone in the static instruction library as the optimal temperature control instruction of the temperature control zone. For example, for each temperature control zone, the processor may determine the reference data closest to various numerical values of the real-time working condition and the target temperature difference range of the temperature control zone as the best matching reference data.

The optimal heating period refers to a minimum duration of heating required to enable the corresponding temperature control zone to achieve the target temperature difference range.

The optimal heating power refers to a minimum heating power required to enable the corresponding temperature control zone to achieve the target temperature difference range.

The optimal cooling period refers to a minimum duration of spraying the cooling gas required to enable the corresponding temperature control zone to achieve the target temperature difference range.

The optimal valve opening refers to a minimum opening of a cooling gas valve required to enable the corresponding temperature control zone to achieve the target temperature difference range.

According to some embodiments of the present disclosure, for each temperature control zone, the molten glass temperature, the material characteristic, the down-draw parameter, and the target temperature difference range corresponding to the temperature control zone are periodically obtained, and the static instruction library is queried to automatically generate a temperature control instruction, thereby achieving dynamic regulation of the temperature of the temperature control zone, which enables fast and precise temperature control of the temperature control zone.

In some embodiments, a plurality of transition zones are provided between the overflow zone, the thickness forming zone, the pre-annealing zone, the soaking zone, the annealing zone, and the subsequent annealing zone, and the method for transverse temperature distribution design of forming and annealing to enhance a draw rate further includes: for each of the plurality of transition zones, determining a temperature transition curve of the transition zone according to target temperature difference ranges of zones on both sides of the transition zone and a length of the transition zone, wherein the temperature transition curve includes target temperature differences at a plurality of position points in the transition zone.

The transition zone refers to a buffer zone jointly established by taking a zone with a certain length from each of two adjacent temperature control zones (e.g., the overflow zone and the thickness forming zone) on both sides of a junction of the two temperature control zones. For example, a zone composed of a zone with a certain length at an end of the overflow zone and a zone with a certain length at a beginning of the thickness forming zone.

In some embodiments, a length of the zone intercepted from each temperature control zone may be preset, and the lengths of the zones intercepted from different temperature control zones may be the same or different. For example, the length of the zone intercepted from each temperature control zone may be positively correlated with a length of the temperature control zone. More descriptions regarding determining the length of the transition zone may be found elsewhere in the present disclosure.

The zones on both sides of the transition zone refer to two temperature control zones adjacent to the transition zone. For example, for a transition zone between the overflow zone and the thickness forming zone, the zones on both sides are the overflow zone and the thickness forming zone.

The length of the transition zone refers to a length of the transition zone in the flow direction. The flow direction refers to a flow direction of the glass substrate, such as the down-draw direction of the glass substrate shown in FIG. 2.

In some embodiments, the length of the transition zone may be set by a technician based on experience. In some embodiments, the length of the transition zone may be determined based on median values of the target temperature difference ranges of two adjacent temperature control zones. For example, the processor may determine two position points corresponding to two median values of the two target temperature difference ranges of the two adjacent temperature control zones as two calibration points, take the two calibration points as a start point and an end point of the transition zone, and determine a length between the two calibration points as the length of the transition zone.

The temperature transition curve refers to a temperature change curve of two adjacent temperature control zones within the transition zone, including target temperature differences at the plurality of position points within the transition zone. In some embodiments, the processor may equally divide the transition zone into a plurality of sub-zones in the flow direction.

In some embodiments, the processor may obtain target temperature differences corresponding to the start point and the end point of the transition zone respectively, and construct the temperature change curve of the transition zone through an interpolation method based on the target temperature differences corresponding to the start point and the end point, respectively. For example, the processor may obtain a median value of the target temperature difference range of a previous temperature control zone of the transition zone (i.e., a first median value) and a median value of the target temperature difference range of a subsequent temperature control zone of the transition zone (i.e., a second median value), and generate the temperature transition curve of the transition zone through the interpolation method based on the first median value and the second median value, using vertical heights of the plurality of position points as horizontal coordinates and target temperature differences as vertical coordinates. For example, the horizontal coordinates from left to right represent vertical heights of the plurality of position points from high to low, and the vertical coordinates represent values of the target temperature differences of the plurality of position points.

The interpolation method includes, but is not limited to, linear interpolation, spline interpolation, etc. In some embodiments, the processor may take a plurality of points at equal intervals in the vertical direction of each sub-zone as data points for interpolation. The count of the plurality of points may be set by a technician based on experience.

In some embodiments, the data points for interpolation may be used as position points, and the temperature differences corresponding to the data points for interpolation may be taken as the target temperature differences.

According to some embodiments of the present disclosure, by providing the transition zone between adjacent temperature control zones and adopting interpolation technology to obtain a smooth temperature transition curve for smooth temperature change control of the transition zone, sudden temperature changes caused by different transverse temperature designs between adjacent temperature control zones are avoided, and the generation of new stress can be prevented.

In some embodiments, the method for transverse temperature distribution design of forming and annealing to enhance a draw rate further includes: for each of the plurality of transition zones, determining a first temperature control instruction and a second temperature control instruction through a first static instruction library and a second static instruction library according to the molten glass temperature, the material characteristic, a down-draw parameter, and the target temperature difference range every preset period; determining a target temperature control instruction based on the first temperature control instruction and the second temperature control instruction, and controlling the plurality of heating units in the transition zone to heat at a target heating power during a target heating period, and/or controlling a plurality of cooling units in the transition zone to spray the cooling gas at a target valve opening during a target cooling period based on the target temperature control instruction.

The first static instruction library refers to a static instruction library corresponding to a previous temperature control zone of the transition zone. The “previous temperature control zone” refers to the temperature control zone located in front among two adjacent temperature control zones according to the flow direction. For example, in a transition zone formed between the overflow zone and the thickness forming zone, the instruction library corresponding to the portion of the transition zone located in the overflow zone is the first static instruction library.

The second static instruction library refers to a static instruction library corresponding to a subsequent temperature control zone of the transition zone. The “subsequent temperature control zone” refers to the temperature control zone located behind among two adjacent temperature control zones according to the flow direction. For example, in the aforementioned transition zone formed between the overflow zone and the thickness forming zone, the instruction library corresponding to the portion of the transition zone located in the thickness forming zone is the second static instruction library.

The first temperature control instruction refers to the optimal temperature control instruction obtained by matching through the first static instruction library.

The second temperature control instruction refers to the optimal temperature control instruction obtained by matching through the second static instruction library.

In some embodiments, for each sub-zone, the processor determines a sub-target temperature difference range based on a plurality of target temperature differences corresponding to the plurality of position points of the sub-zone in the temperature transition curve. For example, the sub-target temperature difference range may be determined based on target temperature differences corresponding to position points at both ends of the sub-zone.

In some embodiments, the processor may obtain the first temperature control instruction and the second temperature control instruction by matching through the first static instruction library and the second static instruction library based on the molten glass temperature, the material characteristic, the down-draw parameter, and a corresponding sub-target temperature difference range of the sub-zone.

The obtaining manner for the molten glass temperature, the material characteristic, and the down-draw parameter of the sub-zone and the determination manner for the first temperature control instruction and the second temperature control instruction are similar to the obtaining manner for the molten glass temperature, the material characteristic, and the down-draw parameter of each temperature control zone and the determination manner for the optimal temperature control instruction. Please refer to the foregoing descriptions related to the optimal temperature control instruction for the temperature control zone.

The target temperature control instruction refers to an instruction for controlling the heating array/the cooling array in the transition zone, including the target heating period, the target heating power, the target cooling period, the target valve opening, etc.

In some embodiments, for each sub-zone, the processor may perform a weighted summation on the first temperature control instruction and the second temperature control instruction and use the temperature control instruction obtained from the weighted summation as the target temperature control instruction.

In some embodiments, weight coefficients for the weighted summation may be set by a technician based on experience. In some embodiments, the weight coefficients for the weighted summation may be related to a distance between the sub-zone and the previous temperature control zone and a distance between the sub-zone and the subsequent temperature control zone, where a closer distance corresponds to a larger weight coefficient. For example, along the flow direction of the glass substrate, a first distance may be determined based on a midpoint of the sub-zone and a midpoint of the previous temperature control zone, a second distance may be determined based on the midpoint of the sub-zone and a midpoint of the subsequent temperature control zone, and then weight coefficients for the first temperature control instruction and the second temperature control instruction may be determined according to magnitudes of the first distance and the second distance. The midpoint of the previous temperature control zone refers to a midpoint of a length of the previous temperature control zone along the flow direction of the glass substrate. The midpoint of the subsequent temperature control zone refers to a midpoint of a length of the subsequent temperature control zone along the flow direction of the glass substrate.

The target heating period, the target heating power, the target cooling period, and the target valve opening are similar to the optimal heating period, the optimal heating power, the optimal cooling period, and the optimal valve opening, respectively. Please refer to the foregoing description related to the optimal temperature control instruction.

According to some embodiments of the present disclosure, by periodically obtaining the molten glass temperature, the material characteristic, the down-draw parameters, and the target temperature difference range of the transition zone, obtaining two temperature control instructions (i.e., the first temperature control instruction and the second temperature control instruction) of the two temperature control zones corresponding to the transition zone through the static instruction libraries of the adjacent temperature control zones, and then determining the target temperature control instruction for the transition zone by performing a weighted summation based on these two temperature control instructions, dynamic regulation of the temperature of the transition zone is achieved, enabling fast and precise temperature control of the transition zone.

According to some embodiments of the present disclosure, by providing the heating array and the cooling array in the annealing furnace and controlling the heating array and the cooling array to perform temperature control on each temperature control zone according to a corresponding target temperature difference range, temperature regulation of each temperature control zone can be achieved more precisely and quickly.

The present disclosure provides a method for transverse temperature distribution design of forming and annealing to enhance a draw rate, which overcomes the limitation of the traditional three-stage model of “melt-viscoplastic body-elastic body” that simply divides glass substrate annealing process into a thickness forming zone, a slow annealing zone, and a rapid annealing zone and cannot reveal the mechanism of the annealing stages. Based on an updated relaxation theory of the glass substrate, starting from six physical flow characteristics (free-flowing melt, high viscoplastic body, elasto-plastic body, initial state of elastomer, sub-rigid body, and rigid body), the forming and annealing of the glass substrate is divided into the overflow zone, the thickness forming zone, the pre-annealing zone, the soaking zone, the annealing zone, and the subsequent annealing zone. Different transverse temperature distributions are applied to the overflow zone, the thickness forming zone, the pre-annealing zone, the soaking zone, the annealing zone, and the subsequent annealing zone to obtain a technical route for designing the transverse temperature distribution in glass forming annealing, and the mechanism of glass annealing is analyzed through the deduction of “temperature difference-structural difference-thermal stress”. The established design concept, method, and process for transverse temperature distribution of forming and annealing to enhance a draw rate, based on the developed relaxation theory, relaxation mechanism, and annealing procedure of the glass substrate, provide a more scientific design method and evaluation standard, ensuring that the glass after annealing and cooling to room temperature has a reasonable warpage magnitude and curved surface shape, and is particularly suitable for the refined design of transverse temperature distribution in the forming annealing of glass substrates with large draw rate, wide plate width, and thin profile.

The present disclosure provides an apparatus for transverse temperature distribution design of forming and annealing to enhance draw rate. The apparatus includes at least one annealing furnace. An interior of the annealing furnace is divided into six zones (i.e., an overflow zone, a thickness forming zone, a pre-annealing zone, a soaking zone, an annealing zone, and a subsequent annealing zone) along a travel direction of a glass substrate. Each zone is provided with an independent temperature control system. The temperature control system is configured to be capable of implementing its corresponding transverse temperature distribution design criterion. More descriptions of the transverse temperature distribution design criterion may be found elsewhere in the present disclosure.

For example, the temperature control system may include a heating element, a temperature sensor, and a controller. The controller is configured to adjust a temperature of each temperature control zone according to a preset temperature distribution curve to ensure that a transverse temperature difference of the glass substrate meets design requirements. Those skilled in the art may understand that the apparatus may further include other auxiliary components, such as a flow guide plate, an edge roller, or a cooling unit, to collaboratively realize the forming and annealing process of the glass substrate.

The apparatus for transverse temperature distribution design of forming and annealing to enhance draw rate provided by the present disclosure not only enables more precise control of the warpage magnitude and curved surface shape of the annealed glass but also improves the overall product quality, and is particularly suitable for the forming annealing treatment of glass substrates with large draw rate, wide plate width, and thin profile that have high difficulty requirements.

The foregoing content is merely for illustrating the technical idea of the present disclosure and cannot be used to limit the protection scope of the present disclosure. Any modifications made based on the technical solution according to the technical idea proposed by the present disclosure shall fall within the protection scope of the claims of the present disclosure.

What is claimed is:

1. A method for transverse temperature distribution design of forming and annealing to enhance a draw rate, the method comprising:
dividing an overflow forming annealing zone of an annealing furnace into a plurality of temperature control zones based on physical flow characteristics of glass in the overflow forming annealing zone in an updated relaxation theory and in combination with glass viscosity corresponding to the physical flow characteristics, wherein types of the plurality of temperature control zones comprise an overflow zone, a thickness forming zone, a pre-annealing zone, a soaking zone, an annealing zone, and a subsequent annealing zone; and
applying different transverse temperature distribution design criterion to the overflow zone, the thickness forming zone, the pre-annealing zone, the soaking zone, the annealing zone, and the subsequent annealing zone, respectively.

2. The method according to claim 1, wherein the method is a real-time transverse temperature distribution design method in a glass forming process, the transverse temperature distribution design criterion comprises a target temperature difference range, and the method further comprises:
for each of the plurality of temperature control zones, determining a type of the overflow forming annealing zone to which the temperature control zone belongs;
determining target temperature difference ranges corresponding to the plurality of temperature control zones according to the transverse temperature distribution design criterion corresponding to types of overflow forming annealing zones to which the plurality of temperature control zones belong; and
controlling a cooling array to spray a cooling gas, and/or controlling a heating array to heat according to the target temperature difference ranges corresponding to the plurality of temperature control zones, to control a temperature difference between a central temperature and an edge temperature in each of the plurality of temperature control zone within the target temperature difference range corresponding to each of the plurality of temperature control zone.

3. The method according to claim 2, wherein controlling the cooling array to spray the cooling gas, and/or controlling the heating array to heat comprises: for each of the plurality of temperature control zones, generating an optimal temperature control instruction through a static instruction library according to a molten glass temperature, a material characteristic, a down-draw parameter, and the target temperature difference range every preset period, wherein the optimal temperature control instruction controls a plurality of heating units in the heating array corresponding to the plurality of temperature control zones to heat at an optimal heating power during an optimal heating period, and/or controls a plurality of cooling units in the cooling array in the plurality of temperature control zones to spray the cooling gas at an optimal valve opening during an optimal cooling period.

4. The method according to claim 2, wherein a plurality of transition zones are arranged between the overflow zone, the thickness forming zone, the pre-annealing zone, the soaking zone, the annealing zone, and the subsequent annealing zone, and
the method further comprises: for each of the plurality of transition zones, determining a temperature transition curve of the transition zone according to a target temperature difference range of zones on both sides of the transition zone and a length of the transition zone, wherein the temperature transition curve comprises target temperature differences at a plurality of position points in the transition zone.

5. The method according to claim 4, wherein the method further comprises:
for each of the plurality of transition zones, determining a first temperature control instruction and a second temperature control instruction through a first static instruction library and a second static instruction library according to a molten glass temperature, a material characteristic, a down-draw parameter, and the target temperature difference range every preset period; and
determining a target temperature control instruction based on the first temperature control instruction and the second temperature control instruction, and controlling a plurality of heating units in the temperature control zone corresponding to the transition zone to heat at a target heating power during a target heating period, and/or controlling a plurality of cooling units in the transition zone to spray the cooling gas at a target valve opening during a target cooling period based on the target temperature control instruction.

6. The method according to claim 1, wherein a physical flow characteristic of the glass in the overflow zone is a free-flowing melt, and a corresponding glass viscosity range is 30K to 160K dPa·s; a physical flow characteristic of the glass in the thickness forming zone is a high-viscosity plastic body, and a corresponding glass viscosity range is 160K to 10⁹ dPa·s; a physical flow characteristic of the glass in the pre-annealing zone is an elasto-plastic body, and a corresponding glass viscosity range is 10⁹ to 10¹² dPa·s; a physical flow characteristic of the glass in the soaking zone is an initial state of elastomer, and a corresponding glass viscosity range is 10¹² to 10¹³ dPa·s; a physical flow characteristic of the glass in the annealing zone is a sub-rigid body, and a corresponding glass viscosity range is 10¹³ to 10^{17.5} dPa·s; and a physical flow characteristic of the glass in the subsequent annealing zone is a rigid body, and a corresponding glass viscosity is at least 10^{17.5} dPa·s.

7. The method according to claim 5, wherein the transverse temperature distribution design criterion of the overflow zone comprises:
based on the physical flow characteristic of the glass in the overflow zone being a free-flowing melt, a transverse temperature distribution of an overflow brick tip of the overflow zone satisfying a formula (1):
$$0 ° C . \leq Δ T 1 = T {1}_{C} - T {1}_{E} \leq 20 ° C . , \qquad ( 1 )$$
wherein in the formula (1), T_{1E} is an edge temperature of the overflow brick tip of the overflow zone; T_{1C} is a central temperature of the overflow brick tip of the overflow zone; T_{1E}≥an upper crystallization temperature T_{XU} of the glass, wherein the upper crystallization temperature T_{XU} of the glass is determined based on a liquidus temperature; and ΔT1 is a transverse temperature difference of the overflow brick tip of the overflow zone.

8. The method according to claim 1, wherein the transverse temperature distribution design criterion of the thickness forming zone comprises:
a transverse temperature distribution of the thickness forming zone along a flow direction of a glass substrate satisfying a formula (2):
$$120 ° C . \leq Δ T 2 = T {2}_{C} - T {2}_{E} \leq 190 ° C . , \qquad ( 2 )$$
setting a range of a transverse temperature difference ΔT2_{C} of a central region of the thickness forming zone to be 0° C. to 20° C. for controlling a uniform temperature distribution of the central region of the thickness forming zone;
wherein in the formula (2), T2_{E} is an edge temperature of the thickness forming zone; T2_{C} is a central temperature of the thickness forming zone; and ΔT2 is a transverse temperature difference of the thickness forming zone along the flow direction of the glass substrate.

9. The method according to claim 1, wherein the transverse temperature distribution design criterion of the pre-annealing zone comprises:
a temperature difference between a central temperature and an edge temperature of the pre-annealing zone gradually decreasing from a theoretical annealing starting point of a glass substrate to an expansion softening point along a flow direction of the glass substrate; and a transverse temperature distribution at the expansion softening point satisfying a formula (3):
$$35 ° C . \leq Δ T 3 = T {3}_{C} - T {3}_{E} \leq 50 ° C . , \qquad ( 3 )$$
wherein in the formula (3), a junction position between the thickness forming zone and the pre-annealing zone is the theoretical annealing starting point of the glass substrate, a junction position between the pre-annealing zone and the soaking zone is the expansion softening point of the glass substrate, T3_{E} is the edge temperature of the pre-annealing zone; T3_{C} is the central temperature of the pre-annealing zone; and ΔT3 is a transverse temperature difference at the expansion softening point.

10. The method according to claim 1, wherein the transverse temperature distribution design criterion of the soaking zone comprises:
a temperature difference between a central temperature and an edge temperature of the soaking zone gradually decreasing from an expansion softening point of a glass substrate to an actual annealing starting point along a flow direction of the glass substrate, and a transverse temperature distribution at the actual annealing starting point satisfying a formula (4):
$$17.5 ° C . \leq Δ T 4 = T {4}_{C} - T {4}_{E} \leq 25 ° C . , \qquad ( 4 )$$
wherein in the formula (4), a junction position between the pre-annealing zone and the soaking zone is the expansion softening point of the glass substrate, a junction position between the soaking zone and the annealing zone is the actual annealing starting point of the glass substrate, T4_{E} is the edge temperature of the soaking zone; T4_{C} is the central temperature of the soaking zone; and ΔT4 is a transverse temperature difference at the actual annealing starting point.

11. The method according to claim 1, wherein the transverse temperature distribution design criterion of the annealing zone comprises:
a temperature difference between a central temperature and an edge temperature of the annealing zone gradually decreasing from an actual annealing starting point of a glass substrate to an actual annealing lower limit point along a flow direction of the glass substrate, and a transverse temperature distribution at the actual annealing lower limit point satisfying a formula (5):
$$0 ° C . \leq Δ T 5 = T {5}_{C} - T {5}_{E} \leq 15 ° C . , \qquad ( 5 )$$
wherein in the formula (5), a junction position between the soaking zone and the annealing zone is the actual annealing starting point of the glass substrate, a junction position between the annealing zone and the subsequent annealing zone is the actual annealing lower limit point of the glass substrate, T5_{E} is the edge temperature of the annealing zone; T5_{C} is the central temperature of the annealing zone; and ΔT5 is a transverse temperature difference at the actual annealing lower limit point.

12. The method according to claim 1, wherein the transverse temperature distribution design criterion of the subsequent annealing zone comprises:
a temperature difference between a central temperature and an edge temperature of the subsequent annealing zone gradually decreasing from an actual annealing lower limit point of a glass substrate to an exit position of the annealing furnace along a flow direction of the glass substrate, and a transverse temperature distribution at the exit position of the annealing furnace satisfying a formula (6):
$$- 35 ° C . \leq Δ T 6 = T {6}_{C} - T {6}_{E} \leq 0 ° C . , \qquad ( 6 )$$
wherein in the formula (6), a junction position between the annealing zone and the subsequent annealing zone is the actual annealing lower limit point of the glass substrate, T6_{E} is the edge temperature of the subsequent annealing zone; T6_{C} is the central temperature of the subsequent annealing zone; and ΔT6 is a transverse temperature difference at the exit position of the annealing furnace.

13. An apparatus for transverse temperature distribution design of forming and annealing to enhance a draw rate, wherein the apparatus is configured to implement the method for transverse temperature distribution design of forming and annealing to enhance the draw rate of claim 1.
